SEQUENCE LISTING (7 sequences)

SEQ ID NO: 1 - DNA, 1336 bp, Bacteroides ovatus
  misc_feature (6)..(6): n is a, c, g, or t
ggtgangacacgngtcctncctgccgataactccgggagancctttcgaaagaannannaataccggatggtttaggaagaccgcatgggagttttagtanggaatttcggttatcgatggggangcgttccnttagtttgttggcggggtaacggcccaccaagactacgatggataggggttctgagaggaaggtcccccacattggaactgagacacggtccaaactcctacgggaggcagcagtgaggaatattggtcaatggncgagagcctgaaccagccaagtagcgtgaaggatganngctctatgggtcgtaaacttcttttatatgggaataaagtattccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgtcactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcnccgcacaagcggaggaacatgtggttttaattcgatgatacgcgaggaanccttacccgggcttaaattgcaacagaatanattggaaacagtatagccgcaaggcaattgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcggctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagc

SEQ ID NO: 2 - DNA, 1396 bp, Bacteroides ovatus
  misc_feature (137)..(137): n is a, c, g, or t
atgcagtcgaggggcagcattttagtttgcttgcaaactgaagatggcgaccggcgcacgggtgagtaacacgtatccaacctgccgataactccgggatagcctttcgaaagaaagattaataccggatggcatantaatatcgcatgatatttttattaaagaatttcggttatcgatggggatgcgttccattagtttgttggcggggtaacggcccaccaagacaacgatggataggggttctgagaggaaggtcccccacattggaactgagacacggtccaaactcanacgggaggcagcagtgaggaatattggtcaatggacgagagcctgaaccagccaagtagcgtgaaggatgaatgctctatgggtcgtaaacttcttttatnatgggaataaagtattccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgtcactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaanccttacccgggcttaaattgcannngaatannntggaaacagtatagccgnaaggcnaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggcgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagc

SEQ ID NO: 3 - DNA, 1395 bp, Bacteroides sp 330163
  misc_feature (882)..(882): n is a, c, g, or t
tgcagtcgaggggcagcattttagtttgcttgcaaactaaagatggcgaccggcgcacgggtgagtaacacgtatccaacctgccgataactcggggatagcctttcgaaagaaagattaatatccgatggtatattaaaaccgcatggttttactattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtattccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagncggaggaacatgtggtttaattcgatgatacgcgaggaacctttacccgggcttaaattgcatttgaataatctggaaacaggttagccgcaaggcaaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggcgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagc

SEQ ID NO: 4 - DNA, 1389 bp, Bacteroides sp 330163
  misc_feature (47)..(50): n is a, c, g, or t
tcgaggggcagcattttagtttgcttgcaaactaaagatggcgaccnnnncacgggtgagtaacacgtatccaacctgccgataactcggggatagcctttcgaaagaaagattaatatccgatggtatattaaaaccgcatggttttactattaaagaatttcggttatcgatggggatgcgttccattagtttgttggcggggtaacggcccaccaagactacgatggataggggttctgagaggaaggtcccccacattggaactgagacacggtccaaactcatacgggaggcagcagtgaggaatattggtcaatggacgagagtctgaaccagccaagtagcgtgaaggatgactgccctatgggttgtaaacttcttttatatgggaataaagtattccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaataatctggaaacagggttagccgcaaggcaaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggcgacaggatgctaatcccaaaaacctctctcagttcggatcgaagtctgcaacccgacttcgtgaagctggattcgctagtaatcgcgcatcagccatggcgcggtgaatacgttcccgggccttgtacannncgcccgtcaagccatgaaagccgggggtacctgaagtacgtaaccgcaaggagc

SEQ ID NO: 5 - DNA, 1401 bp, Bacteroides xylanisolvens
  misc_feature (966)..(966): n is a, c, g, or t
atgcaagtcgaggggcagcattttagtttgcttgcaaactaaagatggcgaccggcgcacgggtgagtaacacgtatccaacctgccgataactcggggatagcctttcgaaagaaagattaatatccgatggtatattaaaaccgcatggttttactattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtattccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgcaactgacactgatgctcgaaagtgtgggtatcaaacaggattagataccctggtagtccacacagtaaacgatgaatactcgctgtttgcgatatacagtaagcggccaagcgaaagcattaagtattccacctgggggagtacgccggcaacggtgaaactcaaaggaattgacgggggcccgcacaagcggaggaacatgtggtttaatttcgatgatacgcgaggaaccttacccgggcttaaatttgcatttgaataatctggaaacnaggttagccgcaaggcaaatgtgaaggtgctgcatggttgtcgtcagctcgtgccgtgnaggtgtcggcttanagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggcgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagc

SEQ ID NO: 6 - DNA, 1376 bp, Bacteroides ovatus
  misc_feature (958)..(958): n is a, c, g, or t
tgcaagtcgagcgatctcttcggagagagcggcggacgggtgagtaacgcgtgggtaacctgccctgtacacacggataacataccgaaaggtatactaatacgggataacatacgaaagtcgcatggcttttgtatcaaagctccggcggtacaggatggacccgcgtctgattagctagttggtaaggtaatggcttaccaaggcaacgatcagtagccgacctgagagggtgatcggccacactggaactgagacacggtccagactcctacgggaggcagcagtggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggaattactgggcgtaaagggtgcgtaggtggttttttaagtcagaagtgaaaggctacggctcaaccgtagtaagcttttgaaactagagaacttgagtgcaggagaggagagtagaattcctagtgtagcggtgaaatgcgtagatattaggaggaataccagtagcgaaggcggctctctggactgtaactgacactgaggcacgaaagcgtggggagcaaacaggattagataccctggtagtccacgccgtaaacgatgagtactaggtgtcgggggttacccccctcggtgccgcagctaacgcattaagtactccgcctgggaagtacgctcgcaagagtgaaactcaaaggaattgacggggacccgcacaagtagcggagcatgtggtttaattcgaagcaacgcgaagaaccttacctaagcttgacatcccactgacctctccctaatcggagatttcccttcggggacagtggtgacaggtggtngcatggttgtcgtcagctcgtgtcgtgagatgttgggttaagtcccgcaacgagcgcaancccttgcctttagttgccagcattaagttgggcactctagagggactgccgaggataactcggaggaaggtggggatgacgtcaaatcatcatgccccttatgcttagggctacacacgtgctacaatgggtggtacagagggttgccaagccgcgaggtggagctaatcccttaaagccattctcagttcggattgtaggctgaaactcgcctacatgaagctggagttactagtaatcgcagatcagaatgctgcggtgaatgcgttcccgggtcttgtacacaccgcccgtcacaccatggaagttgggggcgcccgaagccggttagctaacctttaggaagcggccntcgaag

SEQ ID NO: 7 - DNA, 1367 bp, Bacteroides xylanisolvens
  misc_feature (13)..(14): n is a, c, g, or t
ttggttgcaacgnnagatggcgangggnggnngggtgantnacacgtgtcctacctgccgataactcggggannncctttcgaaagaaanantaatatccgatggtatattaagaccgcatgggnttactattanggaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtattccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaataatctggaaacaggttagccgcaaggcaaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggcgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagc

(19) United States
(12) Patent Application Publication
(10) Pub. No.: US 2011/0076356 A1
(43) Pub. Date: Mar. 31, 2011

(54) Novel Fibro-Biotic Bacterium Isolate

(76) Inventors: Cherie J. Ziemer, Ames, IA (US); Steven Arcidiacono, Bellingham, MA (US); Arthur Ragauskas, Lawrenceville, GA (US); Mark Morrison, Dublin, OH (US)

(21) Appl. No.: 12/569,572

(22) Filed: Sep. 29, 2009

(51) Int. Cl. A23K 1/16; A23K 1/18; C12N 1/20

(52) U.S. Cl. 426/2; 426/61; 435/252.1

(57) ABSTRACT

The present invention relates to an isolated anaerobic bacterium Bacteroides sp. for use as a fibro-biotic for livestock. More specifically, the fibro-biotic supplements a fiber diet to Sus scrofa scrofa with the fibro-biotic improving fiber digestion and decreasing fecal output in these livestock. The present invention also relates to the use of the isolated bacterium as a food composition to improve the health of monogastric livestock and as a feed supplement to livestock.

### FIELD OF INVENTION

The present invention relates to an isolated anaerobic bacterium Bacteroides sp. for use as a fibro-biotic for livestock. More specifically, the fibro-biotic supplements a fiber diet of Sus scrofa scrofa, livestock improving fiber digestion and decreasing fecal output. The present invention also relates to the use of the isolated bacterium as a food composition to improve the health of monogastric livestock and as a feed supplement to livestock.

### BACKGROUND OF INVENTION

The simplest digestive system consists of the mouth, associated glands, esophagus, stomach, small intestine, large intestine, pancreas and liver. This is the type of monogastric gastrointestinal tract is found in the pigs, dogs, mink, fish, monkeys, and humans. A monogastric tract has limited capacity for fiber digestion because of the lack of microbial action at the beginning of the digestive tract as compared to mammals with rumens.

In the United States, there has been high cost related to using corn as livestock feed inasmuch as a portion of corn has been utilized towards ethanol production. As such, there has been an increasing emphasis on substituting a high-fiber cereal feed for corn-based feed for monogastric livestock. However, fiber in the form of non-starch polysaccharides, include cellulose, beta-glucans and arabinoxylans, generally reduces the nutritional value of cereal livestock feeds and is generally considered undesirable as a monogastric livestock feed. When cereals such as wheat and barley are utilized, enzymes can be added to livestock feed to promote digestion of the non-starch polysaccharides. For instance, as disclosed in Emiola et al., 2009, J. Anim Sci., 87:2315-2322 Sus scrofa scrofa that were feed a diet of wheat distillers dried grains with solubles supplemented with a multi-carbohydrase mixture (2,600 units xylanase, 1,200 units glucanase and 1,300 units cellulose per kg feed or 5,200 units xylanase, 2,400 units glucanase and 2,600 units cellulose) exhibited improved growth performance and total tract digestibility than those without the enzymatic supplement.

Pertaining to Sus scrofa scrofa, various diet supplementations have been implemented to confer desirable livestock traits such and increased weight gain or increasing lean-to-fat ration of livestock. To with, U.S. Pat. No. 5,192,804 discloses a feed composition of L-carnitine in an admixture fed to starter Sus scrofa scrofa livestock that increases the lean-to-feed ratio for said Sus scrofa scrofa.

In another example, U.S. Pat. No. 5,077,068 discloses a liquid feed supplement of dry feed grain coated with vegetable fats and aromatic flavoring agents for monogastric animals.

Given the animal feed supplements in the field and an increased emphasis in using high-fiber cereal as a livestock feed source, there is a need to develop a feed supplement increases digestion of fiber material represented by plant cell structures such as lignin, hemicellulose, and cellulose in monogastric livestock. By doing so, the monogastric livestock would be able to more readily able to harness energy from fibrous cereals. Additionally, such a feed supplement would aid in reducing fecal output, thus reducing fecal management and environmental impact costs.

Given the need to utilize high fiber feeds and the limitation of monogastric system there is a need in the art to increase the nutritional value of cereal livestock feeds while maintaining a fiber diet for monogastric livestock.

### BRIEF SUMMARY OF THE INVENTION

Disclosed is an isolated fibro-biotic capable of colonizing and surviving in the gastrointestinal tract of a monogastric animal, wherein the isolate fiber fermenting fibro-biotic is a bacterial strain NRRL B-50315. Also disclosed is a composition comprising a fibro-biotic bacterium, a carrier, and an animal feed, wherein said fibro-biotic bacterium ferments fiber and comprises bacterial isolate NRRL B-50315.

Also disclosed is a method of improving fiber digestion of livestock, the method comprising: preparing a fibro-biotic comprising a live Bacteroides strain; and orally administering said fibro-biotic product to livestock in an amount increasing in vivo fiber digestion. In one embodiment of the invention, the Bacteroides strain is NRRL B-50315. In another embodiment of the invention, the fibro-biotic reduces the fecal output of the livestock. In yet another embodiment of the invention, the livestock is of the genus Sus. A further embodiment of the invention is that the fibro-biotic is administered in conjunction with food or nutritional supplement.

### BRIEF DESCRIPTION OF THE DRAWINGS

The present invention together with the above and other objects and advantages may best be understood from the following detailed description of the embodiment of the invention illustrated in the drawings, wherein:

FIG. 1 is a graph of average fecal output of Sus scrofa scrofa fed either a control fiber diet, high fiber diet, or with both diets combined. For each diet, Sus scrofa scrofa were given either no bacterial feed supplement (indicated as “none”), a feed supplement of Bacteroides strain SD CMC 3f (indicated as “B”), a feed supplement of Bacteroides strain RF Cell 1b2 (indicated as “C”), or a feed supplement of Bacteroides strain SD CC 2a (indicated as “D”).

FIG. 2 is a graph of average carbohydrate digestibility percentage of crude fiber, neutral detergent fiber (hemicellulose+cellulose+lignin content), acid detergent fiber (cellulose+lignin content), and lignin content for Sus scrofa scrofa on a control diet feed diet. In addition to the control feed diet, Sus scrofa scrofa were given either no bacterial feed supplement (indicated as “none”), a feed supplement of Bacteroides strain SD CMC 3f (indicated as “B”), a feed supplement of Bacteroides strain RF Cell 1b2 (indicated as “C”), or a feed supplement of Bacteroides strain SD CC 2a (indicated as “D”).

FIG. 3 is a graph of average carbohydrate digestibility percentage of crude fiber, neutral detergent fiber (hemicellulose+cellulose+lignin content), acid detergent fiber (cellulose+lignin content), and lignin content for Sus scrofa scrofa with both diets combined. In addition to the combined feed diet, Sus scrofa scrofa were given either no bacterial feed supplement (indicated as “none”), a feed supplement of Bacteroides strain SD CMC 3f (indicated as “B”), a feed supplement of Bacteroides strain RF Cell 1b2 (indicated as “C”), or a feed supplement of Bacteroides strain SD CC 2a (indicated as “D”).

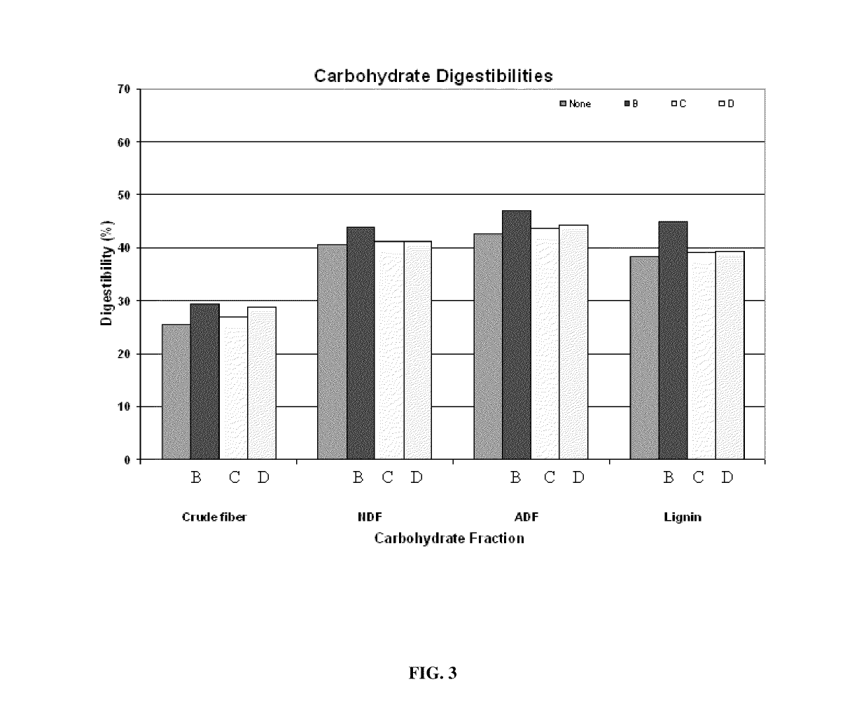

FIG. 4 is a graph of average carbohydrate digestibility percentage of crude fiber, neutral detergent fiber (hemicellulose+cellulose+lignin content), acid detergent fiber (cellulose+lignin content), and lignin content for Sus scrofa scrofa on a high fiber feed diet. In addition to the high fiber feed diet, Sus scrofa scrofa were given either no bacterial feed supplement (indicated as “none”), a feed supplement of Bacteroides strain SD CMC 3f (indicated as “B”), a feed supplement of Bacteroides strain RF Cell 1b2 (indicated as “C”), or a feed supplement of Bacteroides strain SD CC 2a (indicated as “D”).

### DEPOSIT OF BIOLOGICAL MATERIAL

Isolate SD CMC 3f, identified as a strain of Bacteroides ovatus str 4140 based on 16S rRNA gene sequencing. Sequence of SD CMC 3f was 97% similar to the sequences of Bacteroides ovatus strains with the following NCBI Accession numbers: AY895197, DQ100446, AY895193, EU722734, and AY652736. Isolate SD CMC 3f was deposited on Sep. 10, 2009, under the provisions of the Budapest Treaty in the Agricultural Research Culture Collection (NRRL) in Peoria, Ill., and has been assigned Accession No. NRRL B-50315.

### DETAILED DESCRIPTION OF THE INVENTION

### Definitions

As used in the specification and claims, the singular form “a”, “an” and “the” include plural references unless the context clearly dictates otherwise. For example, the term “a bacterium” includes a plurality of unicellular microorganisms of the same species.

As used herein, “monogastric” is intended to encompass any animal having one stomach. Examples of monogastric animals include, but are not limited to, horses, emu, ostrich, dog, cat, swine, bear, turkey, chickens, ducks, quail, pheasants, reptiles, and humans. Pre-ruminant animals such as young cattle, buffalo, bison, and elk are also encompassed by the term monogastric as these animals are born monogastric and then develop into true ruminants as adults. In a preferred embodiment of the invention, the fibro-biotic is administered to livestock that are fed a fiber diet.

As used herein the term fiber refers to the soluble and insoluble components of feed that are not digested by enzymes in the livestock gastrointestinal tract. The primary sources of fiber include such cell wall materials as cellulose, hemicelluloses, lignin, and pectins, along with gums and mucilages from plant material.

The term “crude fiber” is defined as loss on ignition of dried residue remaining after digestion of sample with 1.25 percent sulfuric acid and 1.25 percent sodium hydroxide under specific conditions. Typical conditions and methods are described in the Official Method of Analysis of the Association of Official Analytical Chemists. The undissolved residue is rinsed with dilute sulfuric acid, deionized water, and ethanol, then reduced to ash. The crude fiber value includes the amount of cellulose and some indigestible lignins but does not distinguish between digestible and indigestible fiber.

“Neutral detergent fiber” and “acid detergent fiber” as used herein are analyses that involve the solubilization of non-fiber components of the feed in boiling detergent solution, with the residual material described as neutral detergent fiber or acid detergent fiber depending on the solution used. Neutral detergent fiber (NDF) analysis and acid detergent fiber (ADF) analysis originally described by P. J. Van Soest (Agricultural Handbook No. 379 entitled Forage Fiber Analyses (Apparatus, Reagents, Procedures, and Some Applications); pages 1-20; by H. K. Goering and P. J. Van Soest; Agricultural Research Service of the United States Department of Agriculture) is incorporated herein by reference. Typically, acid detergent fiber is measured by boiling the sample in an acid detergent solution. The mash is filtered. The residue contains wall fiber, primarily cellulose, lignin and silica. Neutral detergent fiber is typically measured by boiling the sample in a neutral detergent solution. The wash is filtered. The residue contains all of the structural plant parts, including cellulose, hemicellulose, and lignin.

As used herein the term “fibro-biotic” refers to live bacteria when fed to a livestock, increases fiber digestion for said livestock.

As used herein the term “isolated bacterium strain” means that the strain might be cultivated in vitro in a culture comprising said strain.

As used herein the term “suitable nutrient medium” means a medium, such as LAB broth, MRS broth, or Wilkens-Calgren broth in which bacteria might be cultivated.

### Bacterial Strain Selection

Initially, 122 bacteria isolates from human excrement was provided from Natick Soldier Center (Natick, Mass.). The 122 isolates were initially sequenced for approximately half of a 16S rRNA gene allowing for grouping by sequence similarity. The isolates were then characterized by api Anaerobe Identification system (bioMérieux, Inc., Lombard, Ill.) and BBL Crystal Anaerobe Identification system (BD Diagnostics, Eden Prairie, Minn.) for their ability to grow on cellulose and xylan. Six bacterial strains (isolates SD CMC 3f, RF Cell 1b2, SD CC 2c, SD CC 1c, SD CC 1b, and RF Cell 1b1) were capable of growing on cellulose or xylan and were identified as Bacteroides ovatus strains. More particularly, strains SD CMC 3f, RF Cell 1b2, SD CC 1c, and SD CC 1b grew on cellulose and SD CC 2c and RF Cell 1b1 grew on xylan. Bacterial strain SD CC2a was characterized as a Bacteroides xylanisolvens strain and also grew on cellulose.

To determine the effectiveness of the six selected bacterial stains, forty-eight female PIC (Pig Improvement Corporation, Lexington, Ky.) grower Sus scrofa scrofa livestock were used to establish the effects feeding fiber utilizing bacteria with standard and high fiber diets. Treatments were arranged as a 2×4 factorial with 2 diets and 4 bacterial treatments. Sus scrofa scrofa (having an initial average body weight of 61.1 kg) were randomly assigned to experimental treatments and housed in two rooms with individual pens at the Iowa State University Swine Nutrition Farm. The Sus scrofa scrofa were fed diet compositions presented in Table 1 and consisted of control (3331 Kcal/kg metabolizable energy, 14% crude protein, 6.3% hemicellulose and 2.7% cellulose) and high fiber (3300 Kcal/kg metabolizable energy, 14% crude protein, 10.4% hemicellulose and 7.7% cellulose). The livestock were fed ad libitum and had free access to water and were adapted to the pens and diets for a period of seven days. All procedures involving animal handling and testing were reviewed and approved by the Iowa State University Committee on Animal Care (approval #9-06-6207-S).

TABLE 1
Diet formulations

| Ingredient | Control (%) | High Fiber (%) |
|---|---|---|
| Corn | 80.90 | 59.89 |
| Soybean meal | 16.69 | 5.37 |
| Distiller's dried grains with solubles | | 20.00 |
| Soybean hulls | | 10.00 |
| Vegetable oil | | 2.3 |
| CaCO₃ | 0.74 | 0.97 |
| Dicalcium phosphate (20% Ca 18.5% P) | 0.93 | 0.35 |
| Salt | 0.35 | 0.35 |
| Vitamin mix | 0.25 | 0.25 |
| Trace mineral mix | 0.05 | 0.50 |
| Selenium premix | 0.05 | 0.50 |
| L-Lysine-HCL | 0.04 | 0.33 |
| L-Threonine | | 0.05 |
| L-Tryptophan | | 0.03 |

While distiller's dried grains with solubles and soybean hulls constitute fiber addition to the animal feed, it is contemplated that other fiber sources such as corn germ meal, wheat middlings, bran from any grain, alfalfa, corn gluten feed, brewer's grain, dried apple pomace, dried citrus pulp, dried citrus peel, sugar beet pulp, soya bean hulls, pectin residue, and other fiber-added sources would be more readily digested with the fibro-biotic.

The bacterial feed treatments consisted of either no bacteria supplement (A) or one of three Bacteroides isolates (B, C, and D) (Table 2). Prior to bacterial treatment and after adaptation, livestock were acclimated to take 20 ml of a 50:50 mixture of food grade glycerol (Sigma) and Wilkens-Chalgren broth orally via a syringe at 0900 daily for two weeks. It is contemplated that other carriers would be suitable to orally deliver the fibro-bacterial. A suitable carrier would encourage the livestock to ingest the carrier with the fibro-bacteria. Ideally, the carrier would be of a viscosity wherein the fibro-bacteria would confer even mixture. In one embodiment of the invention the fibro-biotic is administered with a glycerol carrier.

The fibro-bacteria were grown anaerobically in 100 ml of Wilkens-Calgren broth (Fisher Scientific, Inc, Pittsburg, Pa.) (1 ml of overnight bacterial growth inoculated into 100 ml of broth) for 24 h at 38° C. (final concentration was 1×10⁹ bacterial cells/ml broth). Bacterial doses were prepared each morning by mixing 10 ml of the bacterial culture with 10 ml of sterile anaerobic glycerol (feed grade, Fisher Scientific), dose of bacteria was 1×10¹⁰ bacterial cells. The treatment with no bacteria had broth processed the same way as described above without inoculation of initial broth. Thus control livestock receive the same volume dose but with no bacterial cells in it. In a Coy anaerobic chamber (Coy Laboratory Products, Grass Lake, Mich.) the glycerol-bacteria mixture was placed in sterile 20 ml syringes. Filled syringes were placed, by treatment, into Mitsubishi anaeropak boxes, without gas generator, (Fisher Scientific) and seal prior to transport to the farm. Sus scrofa scrofa livestock were dosed orally at in the morning, daily throughout the experiment.

TABLE 2
Identification of human bacterial isolates capable of growing on cellulose or xylan
using 16S rRNA gene sequencing

| | | Percent | | |
|---|---|---|---|---|
| Isolate^{a} | CHO^{b} | Identity^{c} | Sequence Identity | 16S rRNA sequence |
| SD CMC 3f | Cellulose | 97 | Bacteroides ovatus str | SEQ. ID. No. 1 |
| | | | 4140 | |
| RF Cell 1b2 | Cellulose | 98 | Bacteroides ovatus str | SEQ. ID. No. 2 |
| | | | 4140 or 3941 | |
| SD CC 2c | Xylan | 99 | Bacteroides sp Smarlab | SEQ. ID. No. 3 |
| | | | 3301643 or | |
| | | | Bacteroides sp Smarlab | |
| | | | 3302993 | |
| SD CC 1c | Cellulose | 99 | Bacteroides sp Smarlab | SEQ. ID. No. 4 |
| | | | 3301643 or | |
| | | | Bacteroides sp Smarlab | |
| | | | 3302993 | |
| SD CC1b | Cellulose | 98 | Bacteroides | SEQ. ID. No. 5 |
| | | | xylanisolvens (NCBI | |
| | | | accession no. | |
| | | | AM230650) | |
| RF Cell 1b1 | Xylan | 97 | Bacteroides ovatus str | SEQ. ID. No. 6 |
| | | | 3941 | |
| SD CC 2a | Cellulose | 98 | Bacteroides | SEQ. ID. No. 7 |
| | | | xylanisolvens (NCBI | |
| | | | accession no. | |
| | | | AM230650) | |

^{a}Laboratory isolate identifier.
^{b}Carbohydrate that the strain was originally isolated on.
^{c}Data obtained using Mega-BLAST program (NCBI)

Prior to sampling, three weeks post treatment initiation; 24 pigs at a time were moved into metabolism crates (1.2×2.4 m) for 11 days. On days 7 to 11 total dietary intake, fecal output, and urinary output were measured. On these five days samples were taken of feed and feed refusals and 10% of the fecal output of each pig was pooled in order to determine nutrient digestibilities. Blood was collected from the jugular vein into vacuum containers containing sodium heparin (Becton Dickinson, Franklin Lakes, N.J.) on days 1 and 11 and the resulting plasma was stored at −20° C. until analyzed for plasma energy metabolites and insulin.

Feed, feed refusals, and fecal samples were dried prior to chemical analysis. Carbon, nitrogen and sulfur were analyzed using a VarioMAX CNS analyzer (Elementar Analysensysteme GmbH, Hanau, Germany). Other nutrients were analyzed on feed, feed refusals, and feces by Minnesota Valley Testing Laboratories (New Ulm, Minn.) using AOAC approved methods for ash, crude fiber, acid detergent lignin and crude protein.

Data were analyzed as a 2×2×4 randomized block design with 2 groups of pigs, 2 dietary treatments and 4 bacterial treatments. Statistics were performed using Proc GLM of SAS; no interactions were significant (two-way−group×diet, group×bacteria, diet×bacteria and three way, group×diet×bacteria) so they were removed from the final model.

### EXAMPLE 1

### Fiber Digestion

As detail supra, pigs were fed a fibro-biotic supplement detailed in Table 2 for a period of 36 days. Digestibility of nutrients was determined on feed and pooled fecal sample for each subject. Digestibility of neutral detergent fiber and acid detergent fiber were examined daily. Culture effluent subsamples and feed and inoculum samples were dried overnight in pre-weighed aluminum pans for dry fecal matter determination as is known in the art. NDF and ADF on the feed, inoculum and dried effluents from each culture were determined as detailed supra. Digestibilities (DM, NDF and ADF) were estimated for each culture by calculating total DM, NDF and ADF input and output from total feed weight and total inoculum and effluent volumes. Calculations of percentage of fiber digestibility were calculated as follows:

Percentage of digestibility=[(intake nutrient−fecal fiber)/intake fiber]×100

Intake nutrient was calculated as the percentage of nutrient in the feed multiplied by the grams of feed per day. Fecal fiber was calculated by percentage of nutrient in the feces multiplied by grams of feces per day.

TABLE 3
Effect of fibro-biotics on intake, gain, fecal output, blood
metabolites, and digestibilities in pigs fed a control diet.

| | Treatment | | | |
|---|---|---|---|---|
| | | SD CMC | RF Cell | SD CC |
| | None | 3f | 1b2 | 2a |
| Measure | | | | |
| Feed intake, g/day | 2569.72 | 2510.95 | 2432.05 | 2613.00 |
| Gain, kg/d | 0.81 | 0.86 | 0.74 | 0.96 |
| Fecal output, g/day | 710.68 | 510.97 | 538.07 | 626.43 |
| Fecal Dry Matter output, g/day | 430.67 | 381.01 | 396.23 | 412.05 |
| Gain/intake | 0.33 | 0.34 | 0.30 | 0.38 |
| Feces/intake | | 0.20 | 0.22 | 0.24 |
| Blood metabolites | | | | |
| Glucose, mg/dL | 68.83 | 72.86 | 68.26 | 68.21 |
| Insulin, μg/dL | 0.15 | 0.09 | 0.20 | 0.1 |
| Triglycerides, mg/dL | 17.40 | 21.18 | 19.34 | 19.05 |
| Cholesterol, mg/dL | 66.06 | 79.85 | 78.09 | 76.53 |
| Digestibility, % | | | | |
| Dry matter | 82.61 | 84.01 | 82.37 | 83.76 |
| Crude protein | 81.68 | 82.55 | 81.03 | 82.42 |
| Crude fiber | 19.78 | 23.91 | 16.63 | 22.66 |
| Neutral detergent fiber | 23.48 | 30.77 | 23.40 | 27.91 |
| Acid detergent fiber | 37.94 | 41.32 | 34.63 | 39.04 |
| Starch | 99.68 | 99.33 | 99.50 | 99.54 |
| Lignin | 23.44 | 35.87 | 22.88 | 23.28 |
| Carbon | 81.50 | 82.87 | 81.09 | 82.66 |
| Nitrogen | 78.64 | 79.08 | 77.88 | 79.61 |
| Sulfer | 69.40 | 70.70 | 69.75 | 70.55 |

TABLE 4
Effect of fibro-biotics on intake, gain, fecal output, blood metabolites,
and digestibilities in pigs fed a high fiber diet.

| | Treatment | | | |
|---|---|---|---|---|
| | | SD CMC | RF Cell | SD CC |
| | None | 3f | 1b2 | 2a |
| Measure | | | | |
| Feed intake, g/day | 2488.89 | 2454.73 | 2670.83 | 2744.69 |
| Gain, kg/d | 0.86 | 0.89 | 0.86 | 0.86 |
| Fecal output, g/day | 852.67 | 722.17 | 979.90 | 969.75 |
| Fecal Dry Matter output, g/day | 472.13 | 449.23 | 488.58 | 572.72 |
| Gain/intake | 0.34 | 0.36 | 0.32 | 0.32 |
| Feces/intake | 0.34 | 0.29 | 0.36 | 0.35 |
| Blood metabolites | | | | |
| Glucose, mg/dL | 68.98 | 72.03 | 73.97 | 70.74 |
| Insulin, μg/dL | 0.14 | 0.11 | 0.10 | 0.11 |
| Triglycerides, mg/dL | 28.77 | 25.51 | 24.65 | 26.29 |
| Cholesterol, mg/dL | 94.61 | 98.67 | 85.84 | 83.29 |
| Digestibility, % | | | | |
| Dry matter | 79.27 | 79.73 | 80.09 | 79.50 |
| Crude protein | 74.80 | 76.55 | 76.37 | 76.20 |
| Crude fiber | 30.19 | 33.75 | 36.23 | 33.90 |
| Neutral detergent fiber | 56.14 | 57.90 | 59.90 | 57.40 |
| Acid detergent fiber | 46.99 | 52.07 | 52.37 | 48.83 |
| Starch | 99.42 | 99.52 | 99.45 | 99.55 |
| Lignin | 56.32 | 55.27 | 56.93 | 54.64 |
| Carbon | 78.40 | 78.96 | 79.27 | 78.65 |
| Nitrogen | 74.81 | 76.40 | 80.14 | 75.79 |
| Sulfer | 75.36 | 76.61 | 76.64 | 75.33 |

Plasma glucose concentrations were determined using an enzymatic kit (GAHK20, Sigma Chemical) based on hexokinase activity. Plasma cholesterol and triglycerides were quantified using enzymatic kits (C7510 and T7531, respectively, Pointe Scientific, Canton, Mich.). The intra- and interassay CV for the cholesterol assay were 0.8% and 1.1%, respectively, and the intra- and interassay CV for the triglyceride assay were 1.0% and 2.9%, respectively. Serum insulin concentrations were determined using a porcine-specific insulin ELISA kit (10-1129-01, ALPCO, Windham, N.H.). The insulin ELISA has a range of detection of 0.02 to 1.5 ng/mL and intra- and interassay CV less than 10%.

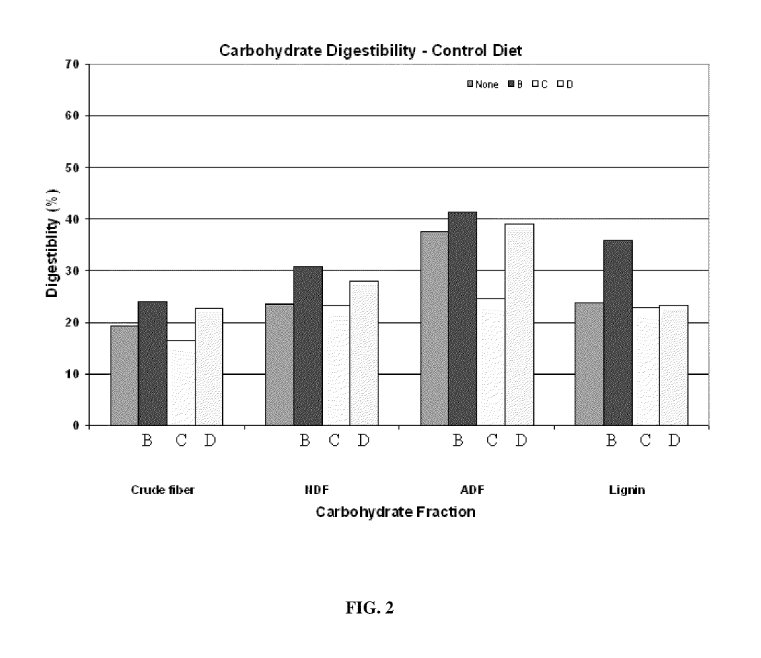

As detailed in FIG. 2, and Table 3, administering Bacteroides strain SD CMC 3f as a feed supplement to Sus scrofa scrofa on a control diet increased digestibility percentage of crude fiber by 4.3 percent when compared to no fibro-biotic supplement. By utilizing SD CMC 3f as a feed supplement in conjunction with a control diet, neutral detergent fiber digestibility increased by 7.29 percent when compared against no fibro-biotic supplement. By utilizing SD CMC 3f as a feed supplement in conjunction with a control diet, acid detergent fiber digestibility increased by 3.38 percent when compared against no fibro-biotic supplement. By utilizing SD CMC 3f as a feed supplement in conjunction with a control diet, lignin digestibility increased by 12.43 percent when compared against no fibro-biotic supplement.

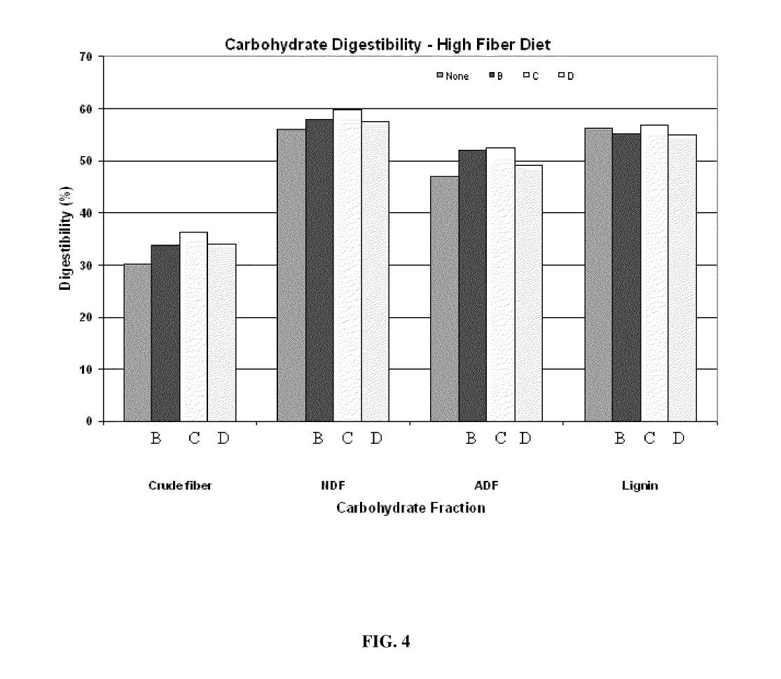

As detailed in FIG. 4, and Table 4, administering Bacteroides strain SD CMC 3f as a feed supplement to Sus scrofa scrofa on a control diet increased digestibility percentage of crude fiber by 3.56 percent when compared to no fibro-biotic supplement. By utilizing SD CMC 3f as a feed supplement in conjunction with a control diet, neutral detergent fiber digestibility increased by 1.76 percent when compared against no fibro-biotic supplement. By utilizing SD CMC 3f as a feed supplement in conjunction with a control diet, acid detergent fiber digestibility increased by 5.08 percent when compared against no fibro-biotic supplement. By utilizing SD CMC 3f as a feed supplement in conjunction with a control diet, surprisingly lignin digestibility decreased by 1.05 percent when compared against no fibro-biotic supplement.

### EXAMPLE 2

### Decreased Fecal Output

As detail supra, Sus scrofa scrofa were fed fibro-biotics detailed in Table 2 for a period of 11 days. Digestibility of neutral detergent fiber and acid detergent fiber were examined daily. Culture effluent subsamples and feed and inoculum samples were dried overnight in pre-weighed aluminum pans for dry fecal matter determination as is known in the art. NDF and ADF on the feed, inoculum and dried effluents from each culture were determined using analytic methods listed supra. Digestibilities (DM, NDF and ADF) were estimated for each culture by calculating total DM, NDF and ADF input and output from total feed weight and total inoculum and effluent volumes.

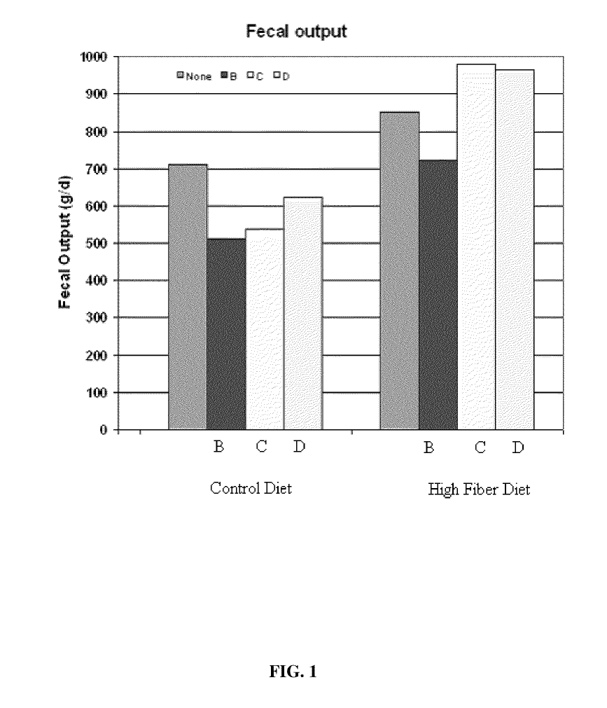

As detailed in FIG. 1 and Table 3 and 4, average fecal output of Sus scrofa scrofa on a control diet supplemented with fibro-biotic B had a decrease in 199.71 grams of daily fecal output compared to those fed only a control diet. With the fibro-biotic B supplement, this was an approximate 39% decrease in daily fecal output when the livestock was on a control diet. Similarly, the average fecal output of Sus scrofa scrofa on a high fiber diet supplemented with fibro-biotic B had a decrease in 130.5 grams of daily fecal output compared to those fed only a high fiber diet. With the fibro-biotic B supplement, this was an approximate 18% decrease in daily fecal output when the livestock was on a high fiber diet.

While the invention has been described with reference to details of the illustrated embodiment, these details are not intended to limit the scope of the invention as defined in the appended claims. The embodiment of the invention in which exclusive property or privilege is claimed is defined as follows:

1. An isolated fibro-biotic capable of colonizing and surviving in the gastrointestinal tract of a monogastric animal, wherein the isolated fiber fermenting fibro-biotic is bacterial strain NRRL B-50315.

2. A composition comprising fibro-biotic bacteria, a carrier and an animal feed, wherein said fibro-biotic bacteria ferments fiber and comprises bacterial isolate NRRL B-50315.

3. The composition of claim 2 wherein the carrier is glycerol.

4. A method of improving fiber digestion of livestock, the method comprising: preparing a fibro-biotic producing comprising a Bacteroides strain; and orally administering said fibro-biotic product to livestock in an amount increasing fiber digestion for said livestock.

5. The method of claim 4 wherein the Bacteroides strain is NRRL B-50315.

6. The method of claim 4 wherein the fibro-biotic reduces the fecal output of the livestock.

7. The method of claim 4 wherein the livestock is of the genus Sus.

8. The method of claim 4 wherein the fibro-biotic is administered in conjunction with food or nutritional supplement.

9. The method of claim 5 wherein approximately 1×10⁹ Bacteroides cells per ml of broth is administered to said livestock.
